(19) United States
(12) Patent Application Publication
(10) Pub. No.: US 2011/0106227 A1
(43) Pub. Date: May 5, 2011

(54) Therapeutic braceletand wrap

(76) Inventors: Marcela N. Desiderio, Bedminster, NJ (US); Ernest D. Buff, Far Hills, NJ (US)

(21) Appl. No.: 12/589,919

(22) Filed: Oct. 30, 2009

(51) Int. Cl. A61F 7/00

(52) U.S. Cl. 607/111

(57) ABSTRACT

A bracelet and wrap having therapeutic properties is worn on a child's injured or sensitive wrist to facilitate healing and/or provide cold/warm treatment. The therapeutic bracelet and warp include a central portion having an internal chamber for receiving and housing a treatment pack. The therapeutic bracelet and warp further include first and second straps including mating fastener means attached to said central portion for securing the bracelet on the child's wrist. The therapeutic pack is appointed to either be a cold treatment pack or a hot treatment pack, depending on the type of treatment needed. Preferably, the treatment pack includes a transparent top wall and a liquid having a plurality of shapes suspended therein. In turn, the central portion's top side is constructed of a transparent material so that the shapes are visible. The therapeutic bracelet and wrap is appointed to be worn on an injury to facilitate healing while providing style, entertainment and distraction to the wearer.

### BACKGROUND OF THE INVENTION

1. Field of the Invention

The present invention relates to a therapeutic bracelet and wrap; and more particularly to a decorative bracelet or wrap having therapeutic thermal and cooling properties with shapes suspended therein and with an optional bandage integrated therein.

2. Description of the Prior Art

Ice packs and heat pads are among the most commonly used treatments in caring for and preventing injuries. Ice treatment is frequently used for injuries to decrease swelling around the injury and help control pain. Ice treatments may also be used for chronic conditions, such as overuse injuries resultant from sports, and game activities, after the activity in order to help control inflammation. On the other hand, heat treatments are recommended for chronic conditions to help relax and loosen tissues, and to stimulate blood flow to the area, before participating in activities. Recommended time for ice and heat treatments is frequently less than twenty minutes at a time. Despite the advantageous and therapeutic nature of heat and ice treatment, children frequently fuss when parents and/or caregivers, or others attempt to place ice or a heat pad on the injury.

While wraps and pads with ice and or heat pads/inserts have been provided, none of these ice/heat pad units provides the ability to entertain the child while the child wears the ice/heat treatment. For examples of the heretofor known and utilized ice wraps, cooling wraps and or heat wraps see: U.S. Pat. No. 4,641,655 to Abt discloses a cooling wrap designed for tying around the neck of a person while performing vigorous activity in hot weather; U.S. Pat. No. 5,476,489 to Koewler discloses a cold therapy system for applying a cooling pad to an area of the body of the user comprising a portable insulated cooler with a lid; U.S. Pat. No. 6,824,556 to Lachance discloses a therapeutic pad system constructed having an ice patch with a generally disc-shaped configuration appointed with a jacket fabricated of a cloth material and has a generally disc shaped configuration with an interior panel and an exterior panel and a pocket there between for receiving the ice patch; U.S. Pat. No. 4,585,003 to Meistrell discloses a heat or cold pack retention device constructed having an elongated, insulative, flexible, relatively thin sheet means having two generally parallel, elongated legs joined to a sheet main portion, the main portion provided with an anchor to anchor the pack, the sheet main portion then engaging and conforming to the shape of the pack, the legs then being adapted to adjustably wrap about and over the same main portion and hold the pack to a user's body; and U.S. Pat. Nos. 5,305,470 and 5,119,513 to McKay discloses a sports band for absorbing perspiration and for cooling the wearer constructed as a band of elastic absorbent material, generally toroidal in shape, configured to be worn upon a person's head or wrist, wherein the band has a slit formed upon its inner surface such that a flexible cold pack may be received thereby and disposed within the band. None of the wraps and pads with ice and or heat pads/inserts provides heat and/or cold treatment constructed as a decorative bracelet with entertainment for children to entice them to wear the treatment.

There is a need in the art for a decorative therapeutic bracelet or wrap appointed to be worn by a person to deliver heat or ice treatments, depending on the nature of treatment. What is more, there remains a need in the art for a therapeutic bracelet or wrap having decorative designs or shapes suspended therein to entice children to wear the bracelet or wrap and receive beneficial hot/cold treatment.

### SUMMARY OF THE INVENTION

The present invention provides a therapeutic bracelet appointed to be worn by a person when its wrist or hand is injured, sensitive and/or necessitates cushioning, protection from dirt and shock absorption, such as during healing. The present invention also provides a therapeutic wrap that can be worn on virtually any part of the body, such as a headband wrap, arm wrap or leg wrap, that is appointed to be worn by a person when he/she is injured, sensitive and/or necessitates cushioning, protection from dirt and shock absorption, such as during healing. Therapeutic properties are achieved by constructing a bracelet or wrap having an internal therapeutic treatment pack that intimately contacts and protects the sensitive or injured part of the body, such as wrist, head, arm, or leg. Although the therapeutic bracelet and wrap herein has particular applications geared toward use on an injured wrist or the like, the therapeutic bracelet or wrap can be utilized on a sensitive part of the body in need of comfort. Gauze pads or bandages may be integrated within the device, which in turn may include medicament.

Generally stated, the therapeutic bracelet includes a central portion having a transparent top side, side walls and a bottom wall constructed with an internal chamber therein that houses a treatment pack. The treatment pack preferably includes a plurality of shapes therein. The bracelet is further constructed having first and second straps including mating fastener means and being attached to the central portion. The therapeutic bracelet is appointed to be worn to provide treatment. Preferably the treatment pack is constructed having a polymeric shell with a cavity housing a liquid therein, which in turn includes a plurality of shapes suspended therein. These shapes are presented in a plethora of designs, including for example, characters, shapes, logos, mythical characters, flowers, airplanes, motor cars, dragons, dolls, soldiers, dancing bears, sharks, fishes, penguins, polar bears, Sponge Bob®, Dora The Explorer®, stars, Disney® characters, and the alike, which, in the cold module become increasingly mobile within the liquid medium as the module progressively transfers its cool temperature to a portion of a person's body in close association therewith, and thaws during that process. A child wearing the device will become enthralled with the shapes or designs, causing the child to continue to keep the ice or heat applied so that proper treatment can be achieved. The shapes also help to take the child's mind off from the pain or injury, in order to provide a better healing experience for the child. The treatment pack may be appointed as a cold pack or as a heat pack, depending on the needs of the user.

In another embodiment, instead of being constructed as a bracelet, a therapeutic wrap is constructed, comprising: (a) a central portion having a transparent top side, side walls and a bottom wall constructed with an internal chamber therein; (b) a treatment pack having a transparent top wall housed within the internal chamber of the central portion; (c) a plurality of shapes integrated within the treatment pack; and (d) first and second straps including mating fastener means and being attached to the central portion. The therapeutic wrap is appointed to be worn to provide treatment. Again, the treatment pack may be constructed as a cold pack or as a heat treatment pack. In this embodiment, the first and second straps are constructed having a longer elongated shape so that same can wrap-around a head and act as a head band, or wrap around a leg or arm and act as a leg or arm band.

Further provided is a method for treating an injury with a therapeutic bracelet, including the step of preparing a treatment pack for hot or cold treatment, the treatment pack containing a plurality of suspended shapes and being housed within an internal chamber of a central portion of a bracelet having first and second straps including mating fastener means and being attached to the central portion. The second step involves placing the therapeutic bracelet on a user's body with the central portion housing the treatment pack being placed in proximity to an injury or sore location on the user's body. Next, the bracelet is secured by way of the first and second straps so that the therapeutic bracelet is secured onto the user's body. While wearing the therapeutic bracelet the child is entertained with the plurality of shapes suspended within the treatment pack during hot or cold treatment. The child continues to wear the therapeutic bracelet for a period of time to facilitate healing treatment. The last step involves removing the therapeutic bracelet after the period of time passes and placing the therapeutic bracelet in a refrigerator/freezer for later cold treatment use or placing the therapeutic bracelet at room temperature until further use is needed.

Lastly, a method for manufacturing a therapeutic bracelet, comprises steps of: (a) forming a bracelet having a central portion and first and second straps; (b) forming an internal chamber within the central portion; (c) integrating mating fastener means within each of the first and second straps; and (d) inserting a treatment pack within the internal chamber of the central portion, the treatment pack having a liquid medium with a plurality of shapes suspended therein and being appointed to deliver cold or hot treatment to a user's body.

The treatment pack and, preferably the central portion, for both the bracelet and/or wrap may be appointed to deliver cold or warm treatment depending on the needs of the wearer. Specifically, ice treatment should be used for abrupt injuries to decrease swelling around the injury and help control pain. Ice treatments should also be used to treat chronic conditions, such as athletic related injuries and game activities, after the activity in order to help control inflammation. In this case, preferably the treatment pack (and visa vie the central portion wherein the treatment pack is integrated within the central portion rather than removable therefrom) and/or the central portion is composed of a transparent rubber (preferably), silicone or polymeric material preferably having a movable fluid therein, such as water, non-toxic water mixtures, or a visco-elastic gel to provide flexibility and heat transfer. Cold therapy is achieved by simply placing the bracelet or wrap (or simply the treatment pack wherein same is removable form the central portion) in a refrigerator or freezer to provide a cold treatment. The treatment pack is constructed having a transparent polymeric top and bottom layer adhered together to form a reservoir or pocket therein which receives and houses a non-toxic material, preferably water, water mixtures, hydroxyethyl cellulose (sold under the trade name Cellusize™) or vinyl-coated silica gel or a granular, vitreous, highly porous form of silica made synthetically from sodium silicate. A plurality of shapes preferably constructed of a polymeric material are located within the reservoir, suspended in the non-toxic liquid material so that the shapes move about when the material is fluid or thawed.

Warm/hot treatment may be provided by the therapeutic bracelet or wrap. Heat treatments are recommended for chronic conditions to help relax and loosen tissues, and to stimulate blood flow to the area, before participating in activities. In another embodiment, the therapeutic bracelet and/or wrap provides a treatment pack appointed to provide warm therapy. Preferably, warm therapy is achieved by way of the treatment pack via a hot water bottle type construct. The heat pack again is to be constructed of a transparent rubber (preferably), silicone or polymeric material having a top and bottom layer adhered together to form a reservoir or pocket therein which receives and houses a non-toxic material, preferably water, water mixtures, hydroxyethyl cellulose (sold under the trade name Cellusize™) or vinyl-coated silica gel or a granular, vitreous, highly porous form of silica made synthetically from sodium silicate. A plurality of shapes preferably constructed of a polymeric material are located within the reservoir, suspended in the non-toxic liquid material so that the shapes move about when the material is fluid or thawed. To heat the pack, the pack may simply be placed in hot water for a few minutes. Alternatively, the treatment pack may be constructed so that is it removable from the central portion. Where the treatment pack is removable, the pack is simply placed in hot water for transfer of heat thereto to provide a warm treatment.

### BRIEF DESCRIPTION OF THE DRAWING

The invention will be more fully understood and further advantages will become apparent when reference is had to the following detailed description of the preferred embodiments of the invention and the accompanying drawings, in which:

FIG. 1a is a schematic view of an embodiment of the therapeutic bracelet and/or wrap wherein a semi-open containing means on one side of the central portion is provided appointed for housing or otherwise containing a sterile gauze pad;

FIG. 1b is a cross-sectional view of the central portion taken along line X-X;

FIG. 2a is a schematic view of another embodiment of the therapeutic bracelet and/or wrap wherein the treatment pack is removable from the central portion;

FIG. 2b is a cross-sectional view of the central portion taken along line Y-Y with the treatment pack being inserted therein;

FIG. 3 is a schematic top-view of another embodiment of the therapeutic bracelet and/or wrap wherein the first and second straps are constructed having elongated cavities therein containing strap treatment mini-packs;

FIG. 4a is a view of an embodiment of the therapeutic wrap appointed to be utilized as a headband, leg band or armband wrap; and

FIG. 4b is a cross-sectional view of the central portion taken along line I-I.

### DETAILED DESCRIPTION OF THE INVENTION

The present invention provides a therapeutic bracelet appointed to be worn by a person when its wrist or hand is injured, sensitive and/or necessitates cushioning, protection from dirt and shock absorption, such as during healing. The present invention also provides a therapeutic wrap that can be worn on virtually any part of the body, such as a headband wrap, arm wrap or leg wrap, that is appointed to be worn by a person when he/she is injured, sensitive and/or necessitates cushioning, protection from dirt and shock absorption, such as during healing. Therapeutic properties are achieved by constructing a bracelet or wrap having an internal therapeutic treatment pack that intimately contacts and protects the sensitive or injured part of the body, such as wrist, head, arm, or leg. Although the therapeutic bracelet and wrap herein has particular applications geared toward use on an injured wrist or the like, the therapeutic bracelet or wrap can be utilized on a sensitive part of the body in need of comfort. Gauze pads or bandages may be integrated within the device, which in turn may include medicament.

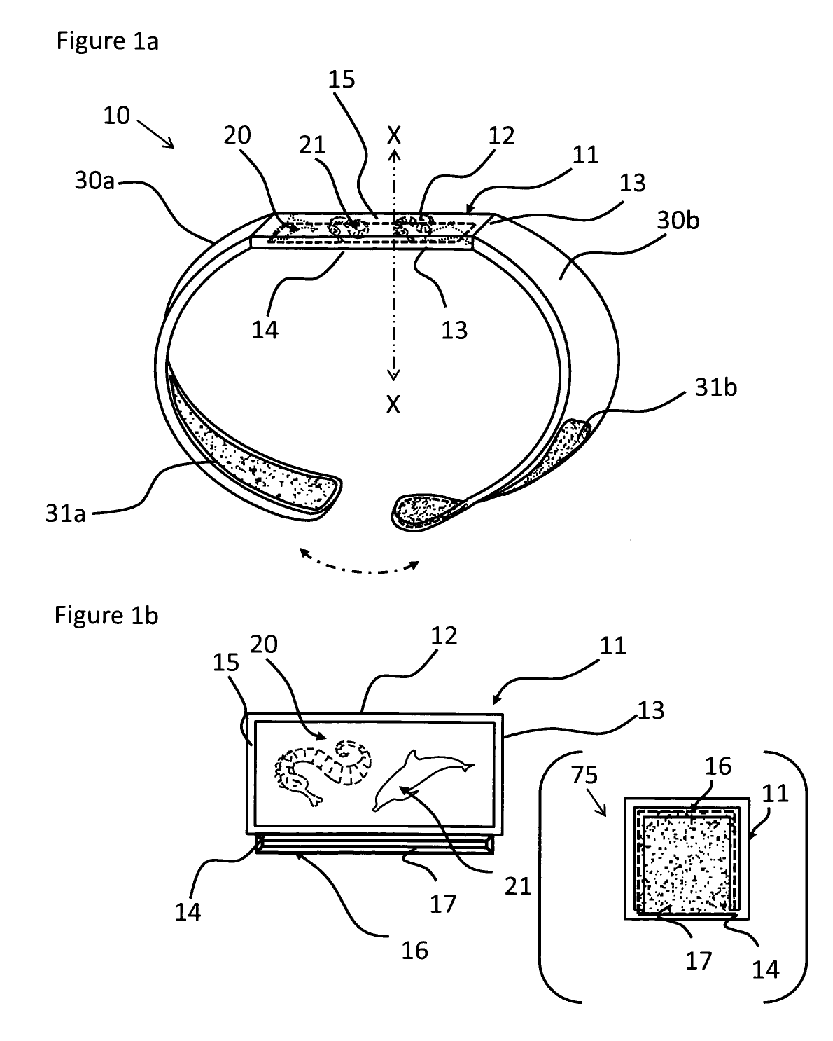

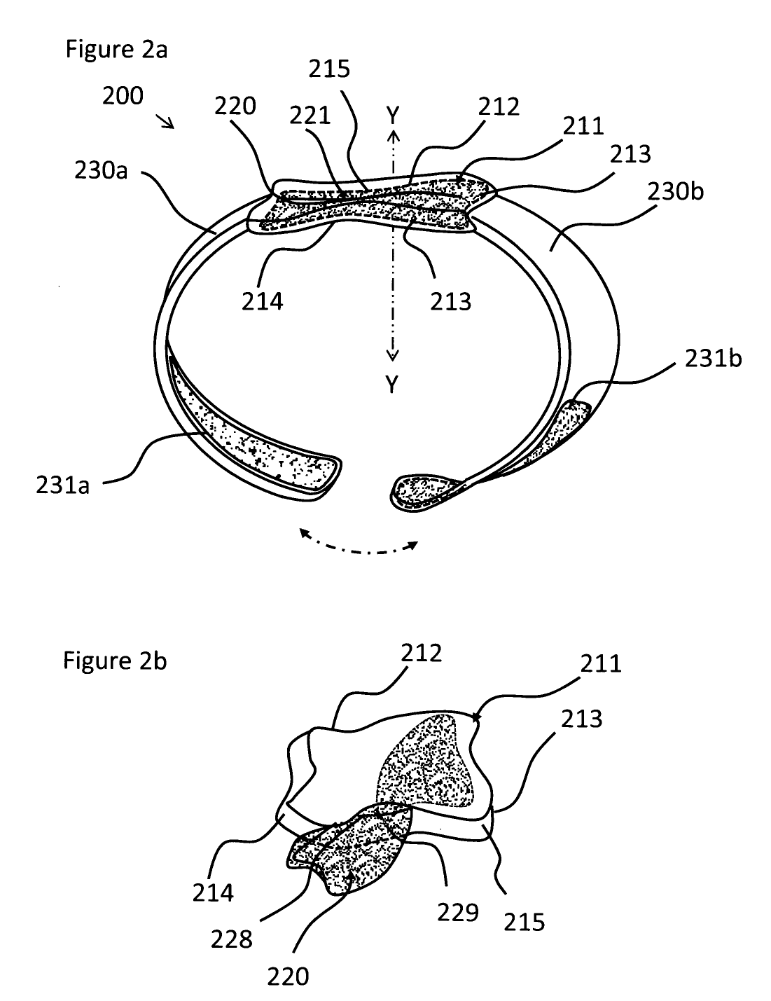

FIGS. 1a-1b illustrate a first embodiment of the therapeutic bracelet, shown generally at 10. The therapeutic bracelet 10 comprises a central portion 11 having a transparent top wall 12, side walls 13 and a bottom wall 14 constructed with an internal chamber 15 therein. A treatment pack 20 is housed within the internal chamber 15 of the central portion 11. This treatment pack 20 may be permanently integrated within the central portion 11, or may be removabley housed within central portion 11 (see FIGS. 2a, 2b). First and second straps 30a, 30b including mating fastener means 31a, 31b are provided attached to opposing side walls 13 of central portion 11. First and second straps 30a, 30b are constructed as elongated portions or arms appointed to wrap around the user's wrist. Where the therapeutic wrap is provided, as opposed to merely a bracelet, the straps are constructed having a longer length so that they can accommodate thicker body portions, such as the leg, arm or head. Where the therapeutic device is a bracelet as herein shown at 10, the length of the straps 30a, 30b preferably each range from 1 inch to 3 inches to accommodate wrist sizes for infants, children and adults; conversely, where the therapeutic device is provided for use as a wrap for the head, legs and/or arms, the length of each strap preferably ranges from 3 inches to 6 inches in length. The width of the straps 30a, 30b may vary, but are preferably of a band type nature, not being too thick in width. Straps 30a, 30b, when in bracelet form, preferably each have a width ranging from 0.25 inches to 2 inches, most preferably being 0.5 inch to 1 inch. This width is generally matched by the central portion 11 as the straps 30a, 30b and central portion 11 are generally equal in width as shown. Central portion 11 preferably has a length ranging from 1 inch to 3 inches; and most preferably is 1 inch to 2 inches in length to optimally accommodate a wrist of a child, or adult. What is more, the treatment pack 20 is only slightly smaller in size than the central portion 11 as it fits snugly within the internal chamber 15 of the central portion 11. Treatment pack 20 is therefore preferably 0.20 inches to 2.85 inches in length; and most preferably is 0.75 inch to 1.75 inches in length.

Mating fastener means 31a, 31b of first and second straps 30a, 30b are preferably constructed as mating hook and loop fasteners such as that sold under the trade name VELCRO®. Optionally, the mating fastener means 31a, 31b may be constructed as mating magnetic portions or snap portions. In any event, the mating fastener means 31a, 31b are provided for quick, easy fastening and quick and easy removal while providing comfortable reliable securement of the bracelet 10 on the user's/child's wrist. Preferably, the first and second straps 30a, 30b are constructed having elastic properties therein so that the first and second straps 30a, 30b are capable of expansion and contraction and being stretched for comfort fit.

Preferably the central portion 11 and visa vie said treatment pack 20 are constructed having a somewhat rectilinear and somewhat curved configuration, deformable and soft in nature to provide the utmost comfort to the wearer. What is more, the therapeutic bracelet 10 is preferably constructed with the treatment pack 20 including a liquid having a plurality of shapes 21 suspended therein. These shapes 21 provide color, design, character, interest and entertainment for children wearing the bracelet 10. What is more, the bracelet 10 is appealing visually and even looks fashionable for the wearer so that it is not merely for therapy, but also is interesting and aesthetically pleasing.

Treatment pack 20 may be provided as a cold treatment pack, hot treatment pack, but is most preferably provided as both a cold and hot treatment pack based upon the storage and use conditions. The treatment pack 20 cooling module/or heat module is constructed as a reservoir containing a liquid “block” medium that can be frozen by refrigeration and re frozen multiple times to provide cooling function, or heated in hot water or even in a microwave (only for a few seconds) to provide a warming function. In operation, the plurality of shaped items 21 become increasingly mobile within the liquid medium as the module progressively transfers its cool temperature to a portion of a person's body in close association therewith, and thaws during that process.

During construction, preferably straps 30a, 30b are constructed of a fibrous material having elastic integrated therein. Straps 30a, 30b are formed as strap like portions and fastening means 31a, 31b are fixedly attached thereto, such as through sowing or adhesive boding, preferably of strips of hook and loop fastening portions such as that sold under the trade name VELCRO®. Straps 30a, 30b are then attached to opposing side walls 13 of central portion 11 by way of heat boding, adhesive boding, or sewing action. Central portion 11 may be constructed in a number of ways, depending on whether the treatment pack 20 is permanently integrated within the central portion 11, or if the treatment pack 20 is removable from the central portion 11. Where the central portion 11 and treatment pack 20 are one unit and the treatment pack 20 is fixedly integrated within the central portion 11, the treatment pack 20 and visa vie central portion 11 are preferably constructed by injection molding formed in the shape of a rectangular with bottom wall 14 being slightly arced. The central portion 11 and treatment pack 20 are preferably constructed of a flexible material so that same conforms comfortably to the shape of the wrist. A transparent first polymeric layer, rubber or silicon layer is formed and is bonded to a second layer by way of adhesive or through heat bonding forming a reservoir or pocket, leaving a small open seem for insertion of liquid into the reservoir or pocket. Bonding may be achieved by an adhesive, RF welding, or any other appropriate means. Plurality shapes 21, preferably constructed from a polymeric, rubber or silicone material and being small in nature, are inserted through the open area of the seam and into the reservoir or pocket. Next, a movable fluid, preferably being distilled water, non-toxic water mixtures, or a visco-elastic gel is injected into the reservoir or pocket and the small open seem and the small open seem is bonded shut, sealing the reservoir or pocket with the fluid and suspended shapes 21 therein. Liquids utilized involve non-toxic material, preferably water, hydroxyethyl cellulose (sold under the trade name Cellusize™) or vinyl-coated silica gel or a granular, vitreous, highly porous form of silica made synthetically from sodium silicate. Cold therapy is achieved by simply placing the bracelet or wrap (or simply the treatment pack wherein same is removable form the central portion) in a refrigerator or freezer to provide a cold treatment. Warm/hot treatment may be provided by way of heating the treatment pack in hot water before use or placing in a microwave for a few seconds prior to use. Preferably, warm therapy is achieved by way of the treatment pack 20 being constructed with distilled water as the liquid contained therein so that is similar to a hot water bottle type warming pack. Preferably, hot water bottle type packs are used where the pack 20 is reusable and where safety is a concern. To heat the pack, the pack may simply be placed in hot water for a few minutes.

Alternatively, the treatment pack 20 may be constructed so that is it removable from the central portion 11. Where the treatment pack 20 is removable, the pack is simply placed in the freezer, refrigerator to provide cold treatment; or in hot water for transfer of heat thereto to provide a warm treatment. Where the treatment pack 20 is removable, the central portion 11 may be constructed of a fabric material, except that it includes a transparent top wall 12 for viewing of the shapes 21. Bottom wall 14 is preferably constructed of a medium capable of heat transfer, while side walls 13 of central portion 11 and top wall 12, and visa vie internal chamber 15, are preferably constructed having an insulating layer. In this manner, heat transfer to the injury is optimized.

In the embodiment shown in FIGS. 1a and 1b, the therapeutic bracelet 10 includes an optional semi-open containing means 16 located on the bottom side 14 of the central portion 11. This semi-open containing means 16 is constructed as a frame pocket having three framed walls and an open wall and an open front wall for insertion of a sterile gauze pad 17 (shown at 75, via bottom view of the central portion 11, semi-open containing means 16, and gauze 17 contained therein shown shaded). Preferably, a plurality of sterile gauze pads 17 are provided with each of the therapeutic bracelets 10 and sold therewith as a kit. What is more, the sterile gauze pads 17 may include medicament or antiseptic ointment integrated therein for direct delivery to the injury to minimize pain.

FIGS. 2a-2b illustrate another embodiment of the therapeutic bracelet, shown generally at 200. Specifically, FIG. 2a is a schematic view of another embodiment of the therapeutic bracelet and/or wrap wherein the treatment pack is removable from the central portion; FIG. 2b is a cross-sectional view of the central portion taken along line Y-Y with the treatment pack being inserted therein. The therapeutic bracelet 210 comprises a central portion 211 having a transparent top wall 212, side walls 213 and a bottom wall 214 constructed with an internal chamber 215 therein. A treatment pack 220 is housed within the internal chamber 215. In this embodiment, the treatment pack 220 is removabley housed within central portion 211. First and second straps 230a, 230b including mating fastener means 231a, 231b are provided attached to opposing side walls 213 of central portion 211. First and second straps 230a, 230b are constructed as elongated portions or arms appointed to wrap around the user's wrist. Mating fastener means 231a, 231b of first and second straps 230a, 230b are preferably constructed as mating hook and loop fasteners such as that sold under the trade name VELCRO®. Preferably the central portion 211 and visa vie said treatment pack 220 are constructed having a somewhat rectilinear and somewhat curved configuration, deformable and soft in nature to provide the utmost comfort to the wearer. What is more, the therapeutic bracelet 210 is preferably constructed with the treatment pack 220 including a liquid having a plurality of shapes 221 suspended therein. These shapes 221 provide color, design, character, interest and entertainment for children wearing the bracelet 210.

In the embodiment shown, the central portion 211 and visa vie internal chamber 15 includes an opening 228 integrated therein so that the treatment pack 220 is removable from the internal chamber 215. The opening 228 is preferably provided as an elongated slit 229 running parallel to the side walls 213 of the central portion 211. Preferably, slit 229 is located on the bottom wall 214 of the central portion 211, or on the side walls 213. Top wall 212 is provided as a transparent wall so that the treatment pack 220 is clearly visible from the outside so that the shapes 221 suspended in the treatment pack 220 are readily visible. Treatment pack 220 may be provided as a cold treatment pack, hot treatment pack, but is most preferably provided as both a cold and hot treatment pack based upon the storage and use conditions. The treatment pack 220 cooling module/or heat module is constructed as a reservoir containing a liquid “block” medium that can be frozen by refrigeration and re frozen multiple times to provide cooling function, or heated in hot water or even in a microwave (only for a few seconds) to provide a warming function. In operation, the plurality of shaped items 221 become increasingly mobile within the liquid medium as the module progressively transfers its cool temperature to a portion of a person's body in close association therewith, and thaws during that process.

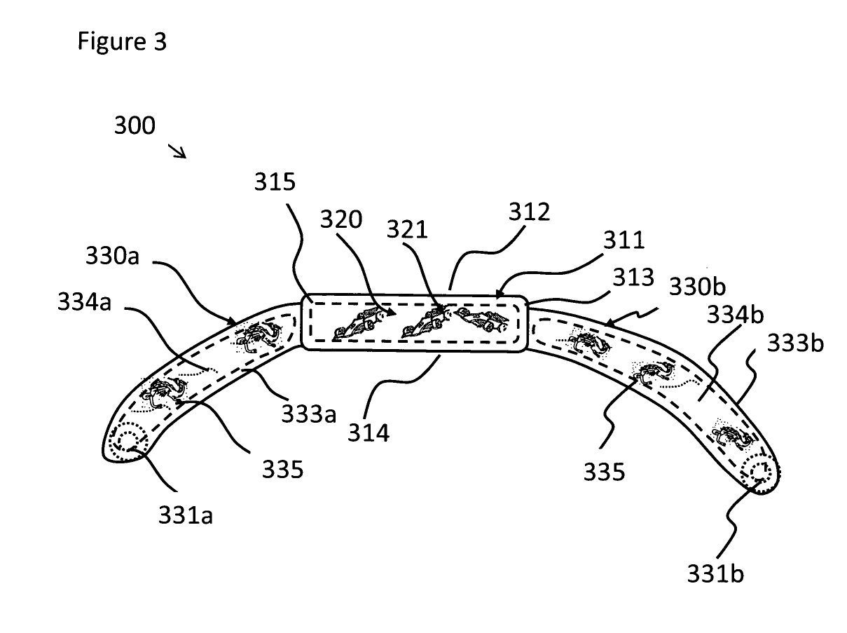

FIG. 3 is a schematic top-view of another embodiment of the therapeutic bracelet and/or wrap wherein the first and second straps are constructed having elongated cavities therein containing strap treatment mini-packs, shown generally at 300. The therapeutic bracelet 310 comprises a central portion 311 having a transparent top wall 312, side walls 313 and a bottom wall 314 constructed with an internal chamber 315 with a treatment pack 320 having shapes 321 suspended in liquid house therein. The treatment pack 320 may be permanently fixed within the internal chamber 315 or may be removabley housed therein. In the embodiment shown, first and second straps 330a, 330b are provided including mating fastener means 331a, 331b constructed as elongated portions or arms appointed to wrap around the user's wrist and have elongated cavities 333a, 333b containing strap treatment mini-packs 334a, 334b therein. In this embodiment, cavities 333a, 333b and visa vie strap treatment mini-packs 334a, 334b is constructed as elongated pockets integrated within the straps 330a, 330b. Elongated pockets 334a, 334b may also contain shapes 335 therein having shapes of character, designs, etc., constructed as the shapes 321 located in treatment pack 320. Preferably, in the straps 330a, 330b are constructed of a polymeric, rubber or silicone material formed with the elongated pocket therein, which is then filled with a liquid as with the treatment pack 320 construction discussed herein. The liquid is preferably water, and the straps 330a, 330b can then be cooled by simply placing the bracelet 300 in the refrigerator or freezer for cold treatment, or placed in hot water for hot treatment.

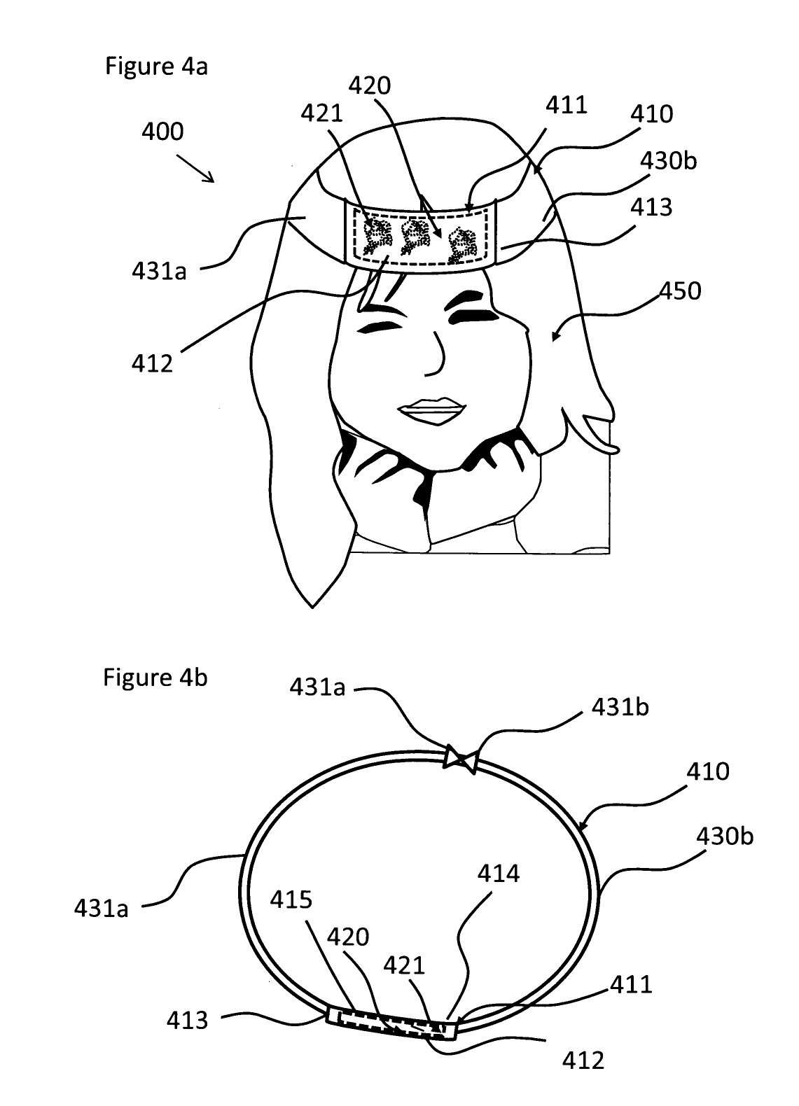

FIGS. 4a-4b illustrate another embodiment of the therapeutic bracelet, shown generally at 400. Specifically, FIG. 4a is a view of an embodiment of the therapeutic wrap appointed to be utilized as a headband, leg band or armband wrap; and FIG. 4b is a cross-sectional view of the central portion taken along line I-I. A therapeutic wrap 410 is provided as a headband. Wrap 410 is constructed having a central portion 411 having a transparent top side 412, side walls 413 and a bottom wall 414 constructed with an internal chamber 415 therein. A treatment pack 420 having a transparent top wall aligned with the transparent top wall 412 of the central portion 411 is housed within the internal chamber 415 of the central portion 411. A plurality of shapes 421 are integrated within the treatment pack 420. The shapes 421 in this use are not visible to the wearer per se, but provide a stylish look to the user for other to view, which in turn will cause the wearer to desire to wear or keep the therapeutic wrap on her head 450, thereby reaping the benefits of the hot/cold treatment. First and second straps 430a, 430b including mating fastener means 431a, 431b are attached to the central portion 411. The therapeutic wrap 410 in the form of a head band is appointed to be worn to provide treatment to a user's head 450 as shown. Because the straps 430a, 430b are elongated in nature and have a long length, the wrap 410 shown could be used on any part of the body with a circumference equal to or less than that of the head 450 shown. For example, the wrap 410 could be used for a leg, or arm, or even torso. Where the therapeutic wrap is provided, as opposed to merely a bracelet, the straps are constructed having a longer length so that they can accommodate thicker body portions, such as the leg, arm or head. Where the therapeutic device is a wrap for the head, legs and/or arms, the length of each strap preferably ranges from 3 inches to 6 inches in length. The width of the straps 430a, 430b may vary, but are preferably of a band type nature, not being too thick in width. Straps 430a, 430b preferably each have a width ranging from 0.25 inches to 2 inches, most preferably being 0.5 inch to 1 inch. This width is generally matched by the central portion 411 as the straps 430a, 430b and central portion 411 are generally equal in width as shown. Central portion 411 preferably has a length ranging from 1 inch to 6 inches; and most preferably is 2 inch to 4 inches in length to optimally accommodate a wrist of a child, or adult. What is more, the treatment pack 420 is only slightly smaller in size than the central portion 411 as it fits snugly within the internal chamber 415 of the central portion 411.

Mating fastener means 431a, 431b of first and second straps 430a, 430b are preferably constructed as mating hook and loop fasteners such as that sold under the trade name VELCRO®. Optionally, the mating fastener means 431a, 431b may be constructed as mating magnetic portions or snap portions. In any event, the mating fastener means 431a, 431b are provided for quick, easy fastening and quick and easy removal while providing comfortable reliable securement of the bracelet 10 on the user's/child's wrist. Preferably, the first and second straps 430a, 430b are constructed having elastic properties therein so that the first and second straps 430a, 430b are capable of expansion and contraction and being stretched for comfort fit. The wrap 410 is constructed similarly to the bracelet discussed hereinabove, save for the length and width of the straps and/or central portion and treatment pack being larger in nature.

Having thus described the invention in rather full detail, it will be understood that such detail need not be strictly adhered to, but that additional changes and modifications may suggest themselves to one skilled in the art. For example, the central portion of the bracelet can be composed of an endothermic material which upon exposure to air becomes cooler; or an exothermic material which becomes warmer upon exposure to air; or a mixture of endothermic and exothermic materials which when exposed to air first become cool and then warm to prevent initial swelling of a wound followed by a warm, pain relieving treatment. Color changes of the liquid contained within the central portion can be triggered by altering impinging light, temperature changes and the like, so that the liquid can be changed from blue to green, or yellow to maroon, for example. Such changes are intended to fall within the scope of the invention as defined by the subjoined claims.

What is claimed is:

1. A therapeutic bracelet, comprising:
a. a central portion having a transparent top side, side walls and a bottom wall constructed with an internal chamber therein;
b. a treatment pack housed within said internal chamber of said central portion;
c. first and second straps including mating fastener means and being attached to said central portion;
wherein said therapeutic bracelet is appointed to be worn to provide treatment.

2. A therapeutic bracelet as recited by claim 1, wherein said treatment pack includes a liquid having a plurality of shapes suspended therein.

3. A therapeutic bracelet as recited by claim 1, wherein said treatment pack is integrated within said central portion.

4. A therapeutic bracelet as recited by claim 1, wherein said treatment pack is removable from said central portion.

5. A therapeutic bracelet as recited by claim 1, wherein said central portion and visa vie said treatment pack are constructed having a somewhat rectilinear and somewhat curved configuration.

6. A therapeutic bracelet as recited by claim 1, wherein said treatment pack is constructed as a cooling module for cold treatment.

7. A therapeutic bracelet as recited by claim 6, wherein said the cooling module contains a liquid “block” medium that can be frozen by refrigeration and re frozen multiple times to provide cooling function.

8. A therapeutic bracelet as recited by claim 7, wherein said cooling module contains a plurality of shaped items appointed to become increasingly mobile within said liquid medium as said module progressively transfers its cool temperature to a portion of a person's body in close association therewith, and thaws during that process.

9. A therapeutic bracelet as recited by claim 1, wherein said treatment pack is constructed as a hot module for hot treatment.

10. A therapeutic bracelet as recited by claim 1, wherein said treatment pack is constructed as a hot/cold module for hot or cold treatment.

11. A therapeutic bracelet as recited by claim 10, wherein said the hot/cold module contains a liquid “block” medium that can be frozen by refrigeration and re frozen or heated multiple times to provide cooling or warming function.

12. A therapeutic bracelet as recited by claim 10, wherein said hot/cold module contains a plurality of shaped items suspended in said liquid medium.

13. A therapeutic bracelet as recited by claim 1 comprising a semi-open containing means on one side of said central portion appointed for housing or otherwise containing a sterile gauze pad.

14. A therapeutic bracelet as recited by claim 13, wherein a plurality of sterile gauze pads are provided with each of said therapeutic bracelets and sold therewith as a kit.

15. A therapeutic bracelet as recited by claim 13, wherein said sterile gauze pads include medicament or antiseptic ointment integrated therein.

16. A therapeutic bracelet as recited by claim 1, wherein said internal chamber within said central portion includes an opening integrated therein so that said treatment pack is removable from said internal chamber.

17. A therapeutic bracelet as recited by claim 1, wherein said first and second straps fastening means are constructed as mating hook and loop fasteners.

18. A therapeutic bracelet as recited by claim 1, wherein said first and second straps are constructed having elastic properties therein so that said first and second straps are capable of expansion and contraction.

19. A therapeutic bracelet as recited by claim 1, wherein said treatment pack comprises polyurethane surrounding a liquid or gel.

20. A therapeutic bracelet as recited by claim 1, wherein at least a portion of said internal chamber of said central portion includes an insulating layer and a transfer layer.

21. A therapeutic bracelet as recited by claim 1, wherein said first and second straps are constructed having elongated cavities therein containing strap treatment mini-packs.

22. A therapeutic wrap, comprising:
a. a central portion having a transparent top side, side walls and a bottom wall constructed with an internal chamber therein;
b. a treatment pack having a transparent top wall housed within said internal chamber of said central portion;
c. a plurality of shapes integrated within said treatment pack;
d. first and second straps including mating fastener means and being attached to said central portion;
wherein said therapeutic wrap is appointed to be worn to provide treatment.

23. A therapeutic bracelet as recited by claim 22, wherein said wrap is a headband.

24. A therapeutic bracelet as recited by claim 22, wherein said treatment pack is a cold treatment pack.

25. A therapeutic bracelet as recited by claim 22, wherein said treatment pack is a heat treatment pack.

26. A method for treating an injury with a therapeutic bracelet, comprising:
a. preparing a treatment pack for hot or cold treatment, said treatment pack containing a plurality of suspended shapes and being housed within an internal chamber of a central portion of a bracelet having first and second straps including mating fastener means and being attached to said central portion;
b. placing said therapeutic bracelet on a user's body with said central portion housing said treatment pack being placed in proximity to an injury or sore location on said user's body;
c. securing said first and second straps so that said therapeutic bracelet is secured onto said user's body;
d. entertaining said user with said plurality of shapes suspended within said treatment pack during hot or cold treatment;
e. wearing said therapeutic bracelet by said user for a period of time to facilitate healing treatment;
f. removing said therapeutic bracelet after said period of time passes and placing said therapeutic bracelet in a refrigerator/freezer for later cold treatment use, or placing said therapeutic bracelet at room temperature until further use is needed.

27. A method for manufacturing a therapeutic bracelet, comprising steps of:
a. forming a bracelet having a central portion and first and second straps;
b. forming an internal chamber within said central portion;
c. integrating mating fastener means within each of said first and second straps;
d. inserting a treatment pack within said internal chamber of said central portion, said treatment pack having a liquid medium with a plurality of shapes suspended therein and being appointed to deliver cold or hot treatment to a user's body.
